(19) United States
(12) Patent Application Publication
(10) Pub. No.: US 2026/0109964 A1
(43) Pub. Date: Apr. 23, 2026

(54) NON-PIEZOELECTRIC FLEXIBLE RESONATOR FOR PROMOTING CELL DIVISION AND MIGRATION

(71) Applicant: INDUSTRY-ACADEMIC COOPERATION FOUNDATION, YONSEI UNIVERSITY, Seoul (KR)

(72) Inventors: Won-gun KOH, Seoul (KR); Hyungsuk LEE, Seoul (KR); Donyoung KANG, Seoul (KR); Byeongseok RYU, Seoul (KR); Sujeong AHN, Seoul (KR); Chanryeol RHYOU, Seoul (KR)

(21) Appl. No.: 19/313,015

(22) Filed: Aug. 28, 2025

(30) Foreign Application Priority Data
Aug. 29, 2024 (KR) 10-2024-0116928

(51) Int. Cl. C12N 13/00; C12M 1/42

(52) U.S. Cl. CPC C12N 13/00; C12M 35/04

(57) ABSTRACT

An embodiment relates to a resonator that promotes cell division and migration by depositing an interdigitated electrode structure on a surface of a dielectric polymer substrate and generating an acoustic wave when an alternating current corresponding to a resonant frequency is applied.

### CROSS REFERENCE TO RELATED APPLICATION

The present application claims priority to Korean Patent Application No. 10-2024-0116928, filed on Aug. 29, 2024, the entire contents of which are incorporated here for all purposes by this reference.

### Technical Field

The present invention relates to an acoustic wave generating resonator, wherein an interdigitated electrode structure is deposited on a surface of a non-piezoelectric dielectric polymer substrate to generate an acoustic wave when an alternating current corresponding to a resonant frequency is applied, thereby promoting cell division and migration.

### Related Art

In the field of tissue engineering, cell stimulation is gaining attention as a new technology that can contribute to three-dimensional tissue development. A cell stimulus delivered from an external source promotes cell division, migration, and differentiation by activating specific signaling pathways or modulating the intracellular microenvironment.

Among various source technologies for cell stimulation, chemical and electrical methods are generally used, but they have the following clear limitations when applied in real life; chemical stimulation is based on the movement of a substance to a target point entirely through the phenomenon of chemical diffusion, so there is a risk of diffusion to unwanted areas; in the case of electrical stimulation, the stimulation efficiency is low due to the low conductivity of tissues in the body.

To overcome these limitations, a cell stimulation method using acoustic waves is newly emerging in the tissue engineering field. Cell stimulation using acoustic waves has the following advantages; it has excellent cell stimulation efficiency, high applicability to the inside of the body, and is non-invasive. In addition, since the intensity of the signal and other parameters can be simply adjusted, it has few side effects and exhibits excellent biocompatibility.

As such, in order to stimulate cells through acoustic waves, a resonator that can cause resonance at a specific frequency is required, and the majority of resonator are manufactured based on piezoelectric elements. Representative piezoelectric elements include Lithium niobate(LiNbO₃), Lead zirconate titanate(PZT), and Zinc oxide (ZnO). However, since these are inorganic-based brittle materials, they exhibit limited biocompatibility and are not flexible, making them difficult to attach to curved tissues and skin, thus limiting their application in the biomedical field. Polyvinylidene fluoride (PVDF) is a representative piezoelectric polymer, and when it is used as a substrate for a resonator, biocompatibility, flexibility, and transparency can be secured, but its electromechanical coupling efficiency is lower than that of ceramics, so additional processes and manpower are required to increase it, which entails enormous costs.

Thus, in order to develop a wearable electronic device type resonator, technology that can secure biocompatibility, flexibility, and transparency while having excellent performance is additionally required. A conventional general resonator is based on the principle that a surface acoustic wave is generated from a substrate by the inverse piezoelectric effect when an electrode with an interdigitated pattern is coated on the surface of a piezoelectric element and an alternating current with a frequency that can cause constructive interference corresponding to the electrode width is applied.

However, this electromechanical coupling phenomenon can also be realized with a dielectric material without piezoelectricity and is based on the polarization that occurs in the dielectric material when an electric field is applied and the mechanical deformation resulting therefrom. In reality, there is no platform that has implemented resonance in the megahertz frequency range using a non-piezoelectric dielectric polymer as a substrate.

### DISCLOSURE OF INVENTION

### Technical Problem

The healing method through ultrasound has the advantage that there are few side effects when acoustic waves generated within an appropriate intensity and frequency are administered to a patient, and the therapeutic effect can be maximized by using it in parallel with existing drug treatment. This is a non-invasive method in which acoustic waves generated from an Ultrasound probe are transmitted from the skin to the tissue to promote the proliferation, migration, or differentiation of cells within the healing site, and the treatment efficiency is excellent because attenuation by the medium is small during the transmission of acoustic waves.

Chronic wounds, etc., caused by chronic diseases such as diabetes require continuous management and healing, but there is a clear limit to equipping such ultrasound healing facilities in a general household and deploying professional manpower. To overcome this, research on the development of patch-type wearable resonators that can generate acoustic waves is being actively conducted. However, the majority of resonators are manufactured based on piezoelectric elements in the form of inorganic ceramics, and since ceramic is a brittle material, its attachment to a curved surface is limited. In addition, continuous friction and pressure can be applied to the tissue due to mechanical mismatch caused by the difference in physical properties between the tissue or skin and the device, which can be a potential factor causing skin necrosis. In addition, the low biocompatibility of inorganic materials can cause immune reactions such as rashes, edema, and inflammation.

The wearable electronic device patch according to the present invention means a device that can be attached to the skin or tissue for use. For this, biocompatibility, transparency, and flexibility must be secured from the substrate of the device, but the existing resonator using a piezoelectric ceramic as a substrate is bound to be limited in these matters due to the characteristics of the substrate itself.

Therefore, an object of the present invention is to provide a dielectric polymer-based resonator that can replace the existing piezoelectric element-based resonator.

In addition, an object of the present invention is to provide a new platform that can overcome the shortcomings of existing resonators by maximizing the selectivity of materials used as a substrate of the resonator and providing biocompatibility, flexibility, and transparency.

### Technical Solution

The present invention provides an acoustic wave generating resonator for cell stimulation, comprising: a non-piezoelectric dielectric polymer substrate;

an IDT thin film formed on one surface of the substrate and comprising an Interdigitated transducer (IDT) electrode and a pad electrode electrically connected to the IDT electrode;

an insulating film formed on the one surface of the substrate and housing the IDT thin film therein;

and a ground thin film formed on the other surface of the substrate, wh an electric field is formed across the substrate by an alternating current voltage applied between the IDT thin film and the ground thin film, and polarization of the substrate induced by the electric field causes mechanical deformation to generate an acoustic wave.

The material of the non-piezoelectric dielectric polymer substrate may be Polyimide (PI), Polyethylene terephthalate (PET), Polydimethylsiloxane (PDMS), or Polyurethane (PU).

The thickness of the non-piezoelectric dielectric polymer substrate may be 5 to 150 μm.

The IDT thin film may be composed of a metal or a conductive polymer, the metal may be one or more selected from the group consisting of gold (Au), copper (Cu), aluminum (Al), and Indium Tin Oxide (ITO), and the conductive polymer may be PEDOT: PSS.

The thickness of the IDT thin film may be 10 to 200 nm.

The IDT electrode and the pad electrode may be respectively composed of a signal electrode and a ground electrode, the signal electrode of the IDT electrode may be electrically connected to the signal electrode of the pad electrode, and the ground electrode of the IDT electrode may be electrically connected to the ground electrode of the pad electrode.

The IDT electrode and the pad electrode may respectively exist as an even number of two or more, and the IDT electrode and the pad electrode may exist in the same number.

In the IDT electrode, one signal electrode may comprise one or more fingers, one ground electrode may comprise one or more fingers, and one finger of the signal electrode may have a structure interdigitated with one finger of the ground electrode.

The length of the one finger may be 5 to 15 mm, and the width may be 50 to 100 μm.

The distance between the one finger and an adjacent finger may be 30 to 500 μm.

The insulating film may comprise one or more selected from the group consisting of Polydimethylsiloxane (PDMS), Polyurethane (PU), and Silicone elastomer.

The ground thin film may be composed of a metal or a conductive polymer, the metal may be one or more selected from the group consisting of gold (Au), copper (Cu), aluminum (Al), and ITO, and the conductive polymer may be PEDOT: PSS.

The thickness of the ground thin film may be 10 to 200 nm.

The resonator may generate resonance by the occurrence of constructive interference along with wave generation when an alternating current having a frequency of 1 to 100 MHz is applied.

The acoustic wave generated from the resonator may promote cell migration and Proliferation.

The resonator may generate an acoustic wave amplified by constructive interference when an alternating current having a frequency in a range of 1 to 100 MHz is applied.

The resonator may have flexibility, enabling attachment to a curved surface of a human body, and have transparency, enabling real-time optical imaging of cells cultured on the device. In addition, the present invention provides a method for manufacturing a resonant device for acoustic wave generation, comprising: a step of preparing a non-piezoelectric dielectric polymer substrate;

In addition, the present invention provides a method for manufacturing an acoustic wave generating resonator, comprising:
a step of preparing a non-piezoelectric dielectric polymer substrate;
a step of depositing an IDT thin film, comprising an Interdigital transducer (IDT) electrode and a pad electrode electrically connected to the IDT electrode, on one surface of the substrate so as to form an electric field with the substrate interposed therebetween;
a step of coating an insulating film on the one surface of the substrate so as to house the IDT thin film therein; and
a step of depositing a ground thin film on the other surface of the substrate.

In addition, the present invention provides an in vitro method for cell stimulation using the aforementioned resonant device for acoustic wave generating resonator.

### Advantageous Effects

The present invention provides an acoustic wave generating resonator composed of a system in which a non-piezoelectric dielectric polymer substrate and an IDT electrode are deposited. The acoustic wave generating resonator for acoustic wave generation according to the present invention can present a new platform that can simulate the surface acoustic wave generation mechanism attributed to the conventional piezoelectric effect by using a non-piezoelectric material.

The resonant frequency of the wave that can be implemented in the present invention is in the megahertz (MHz) band, which can provide an appropriate stimulation effect to cells, and this surpasses the maximum frequency range that can be implemented with a conventional dielectric actuator. The wave generated from the resonator of the present invention can have an Acoustic intensity that can stimulate cells with an applied voltage at the millivolt (mV) level.

The manufacturing process of the resonator of the present invention consists of two steps, an electrode thin film deposition process and a spin coating process, so it is easy to manufacture and can minimize the required process cost.

The resonator of the present invention has superior biocompatibility compared to a conventional piezoelectric element-based resonator wearable patch, so side effects including inflammatory reactions can be minimized. In addition, since the resonator is based on a flexible and transparent dielectric polymer material, it can provide the advantage of being easy to attach to a curved skin/organ surface and enabling real-time imaging.

### BRIEF DESCRIPTION OF THE DRAWINGS

FIG. 1 is a schematic diagram showing a manufacturing process of an acoustic wave generating resonator of the present invention.

FIG. 2 is an operational schematic diagram of the acoustic wave generating resonator of the present invention.

FIGS. 3 and 4 show a schematic diagram of an IDT thin film of the present invention and a manufactured IDT thin film.

FIG. 5 is data showing the reproducibility of the manufacturing process of the acoustic wave generating resonator of the present invention.

Specifically, (a) is a micrograph showing the uniformity of the deposited IDT electrode and the connection between the pad electrode and the IDT electrode, and (b) shows the finger width measurement results.

FIG. 6 is data analyzing the transparency of the acoustic wave generating resonator of the present invention (a) qualitatively and (b) quantitatively.

FIG. 7 is data confirming the flexibility of the acoustic wave generating resonator of the present invention.

Specifically, \textcircled{1} is a photograph of the resonator to which a deformation of 2.5 mm is applied by a UTM, \textcircled{2} is an electron micrograph of the IDT electrode (low magnification), \textcircled{3} is an electron micrograph of the IDT electrode (high magnification/before deformation experiment—control group), and \textcircled{5} is an electron micrograph of the IDT electrode (high magnification/after deformation experiment-experimental group).

FIG. 8 is data confirming the resonance phenomenon of acoustic waves generated from the acoustic wave generating resonator of the present invention.

Specifically, (a) is the streaming tracking path of polystyrene beads, and (b) is the streaming velocity analysis result of the polystyrene beads.

FIG. 9 is the streaming velocity analysis result of polystyrene beads according to the electrode connection structure (ground position).

FIG. 10 is data showing that a resonance phenomenon occurs in a dielectric polymer other than polyimide.

Specifically, it is a bead streaming analysis graph and photographs of resonators manufactured using PET and PDMS.

FIG. 11 is data confirming the promotion of cell migration and proliferation when cells are stimulated with acoustic waves.

Specifically, (a) shows a cell experiment set-up and schematic diagram, (b) shows a duty ratio schematic diagram for pulsed wave application, (c) shows toxicity evaluation (Live/Dead staining fluorescence imaging) results, and (d) and (e) show the evaluation results of wound healing rate and cell proliferation rate due to acoustic waves.

FIG. 12 shows the analysis results of the acoustic wave generation aspect (change in resonant frequency) of the resonators according to the width of the finger.

### DETAILED DESCRIPTION

The present invention relates to an acoustic wave generating resonator for cell stimulation, and the acoustic wave generating resonator for cell stimulation according to the present invention may comprise: a non-piezoelectric dielectric polymer substrate;
an IDT thin film formed on one surface of the substrate and comprising an Interdigitated transducer (IDT) electrode and a pad electrode electrically connected to the IDT electrode;
an insulating film formed on the one surface of the substrate and housing the IDT thin film;
and a ground thin film formed on the other surface of the substrate, wherein an electric field is formed across the substrate by an alternating current voltage applied between the IDT thin film and the ground thin film, and polarization of the substrate induced by the electric field causes mechanical deformation to generate an acoustic wave.

Hereinafter, the acoustic wave generating resonator for cell stimulation for acoustic wave generation of the present invention will be described in detail.

In one specific embodiment, the material of the non-piezoelectric dielectric polymer substrate may be Polyimide (PI), Polyethylene terephthalate (PET), Polydimethylsiloxane (PDMS), or Polyurethane (PU).

The non-piezoelectric dielectric polymer is an insulator and a dielectric, can be used as a substrate of a resonator, and can secure biocompatibility, flexibility, and transparency all at once. The non-piezoelectric dielectric polymer can replace conventional piezoelectric ceramics or polymers and be applied to a wearable electronic device resonator, thereby maximizing the selectivity of materials. In addition, the excellent biocompatibility, flexibility, and transparency of the substrate enable minimized inflammatory reactions, easy attachment to curved surfaces, and real-time imaging, respectively, and can overcome the clear disadvantages of ceramic-based devices. Furthermore, since non-piezoelectric dielectric polymers are very inexpensive compared to piezoelectric ceramics or polymers and can be easily purchased on the market, they can be provided to consumers at a reasonable price during prototype production, and the production cost can be drastically reduced. For example, since PET, etc., are recyclable materials, they can provide eco-friendly advantages when used as a substrate.

In one specific embodiment, the thickness of the non-piezoelectric dielectric polymer substrate may be 5 to 150 μm or 9 to 100 μm. It is possible to secure biocompatibility, flexibility, and transparency within the above thickness range.

In the present invention, the IDT thin film is formed on one surface of the non-piezoelectric dielectric polymer substrate. The IDT thin film comprises an Interdigitated transducer (IDT) electrode and a pad electrode electrically connected to the IDT electrode.

In one specific embodiment, the IDT thin film, i.e., the IDT electrode and the pad electrode, may be composed of a metal or a conductive polymer. The type of the metal is not particularly limited, and may be one or more selected from the group consisting of gold (Au), copper (Cu), aluminum (Al), and Indium Tin Oxide (ITO). In addition, the type of the conductive polymer is not particularly limited, and may be PEDOT: PSS. Specifically, the IDT thin film may be composed of a metal, and preferably may be composed of gold.

The IDT electrode and the pad electrode may be composed of the same material.

In one specific embodiment, the thickness of the IDT thin film may vary depending on the material of the thin film, and may be 10 to 200 nm. If this range is exceeded, the desired transparency cannot be secured. For example, when ITO is used as the material of the thin film, the thickness may be 100 to 200 nm, and when gold is used, the thickness may be 10 to 20 nm.

In one specific embodiment, the IDT electrode may be composed of a signal electrode and a ground electrode. In addition, the pad electrode may be composed of a signal electrode and a ground electrode. That is, the signal electrode of the IDT electrode may be electrically connected to the signal electrode of the pad electrode. In addition, the ground electrode of the IDT electrode may be electrically connected to the ground electrode of the pad electrode.

In one specific embodiment, the structure of the IDT electrode and the pad electrode may be designed to have a structure optimized for acoustic wave generation.

In one specific embodiment, the number of IDT electrodes and pad electrodes may each be an even number of two or more. The IDT electrodes and pad electrodes exist in the same number, and may each be four. For example, if there are four IDT electrodes and four pad electrodes, two IDT electrodes and pad electrodes may be signal electrodes, and two IDT electrodes and pad electrodes may be ground electrodes.

In one specific embodiment, the even number of pad electrodes may be positioned to face each other, and specifically, a ground electrode may be located at a position facing a signal electrode.

In one specific embodiment, in the IDT electrode, one signal electrode may be composed of one or more, or 1 to 50 fingers, and one ground electrode may be composed of one or more, or 1 to 50 fingers. In addition, one finger of the signal electrode may have a structure interdigitated with one finger of the ground electrode.

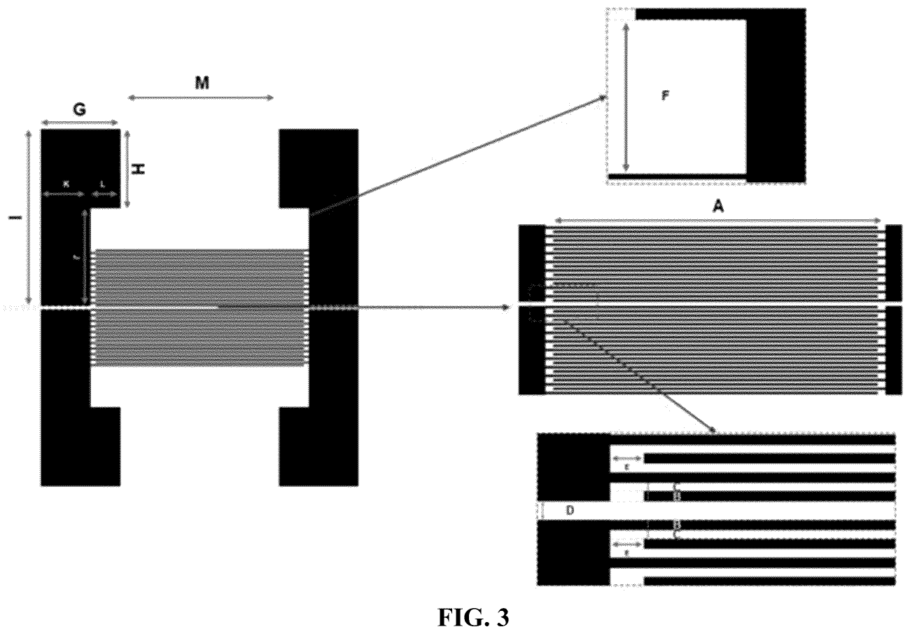

FIG. 3 of the present invention shows an IDT thin film structure according to an exemplary embodiment of the present invention. In the figure, the pad electrode on the upper left represents a signal electrode (hereinafter, first pad-signal electrode), the pad electrode on the upper right represents a ground electrode (hereinafter, first pad-ground electrode), the pad electrode on the lower left represents a ground electrode (hereinafter, second pad-ground electrode), and the pad electrode on the lower right represents a signal electrode (hereinafter, second pad-signal electrode). In addition, the IDT electrodes electrically connected to the pad electrodes are, in order, a first IDT-signal electrode, a first IDT-ground electrode, a second IDT-ground electrode, and a second IDT-signal electrode.

In one specific embodiment, one end of the finger of the first IDT-signal electrode is connected to the first pad-signal electrode. The other end of the finger is located with a gap (E) from the first pad-ground electrode. And, one end of the finger of the first pad-ground electrode is connected to the first pad-ground electrode. The other end of the finger is located with a gap (E) from the first IDT-signal electrode.

In one specific embodiment, the length (A) of one finger may be 5 to 15 mm, and the width (B) may be 50 to 100 μm.

In one specific embodiment, the distance (C) between one finger and an adjacent finger may be 30 to 500 μm.

In one specific embodiment, the gap (E) between the other end of one finger and the pad electrode may be 0.1 to 1 mm.

In the present invention, the insulating film is formed on one surface of the substrate and houses the IDT thin film. In the present invention, the IDT thin film can be protected by forming an insulating film to house the IDT thin film.

In one specific embodiment, the insulating film may comprise one or more selected from the group consisting of Polydimethylsiloxane (PDMS), Polyurethane (PU), and Silicone elastomer.

In one specific embodiment, the thickness of the insulating film may be 1 to 50 μm. In this case, the thickness of the insulating film means the thickness from the non-piezoelectric dielectric polymer substrate to the surface where the insulating film begins.

In the present invention, the ground thin film is formed on the other surface of the substrate. In the present invention, resonance can be generated through constructive interference with waves that occur when an alternating current with a specific frequency is applied to the repeating pattern of signal and ground electrodes.

In one specific embodiment, the ground thin film may be composed of a metal or a conductive polymer. The type of the metal is not particularly limited, and may be one or more selected from the group consisting of gold (Au), copper (Cu), aluminum (Al), and Indium Tin Oxide (ITO). In addition, the type of the conductive polymer is not particularly limited, and may be PEDOT: PSS. Specifically, the ground thin film may be composed of a metal, and preferably may be composed of gold. More specifically, the ground thin film may have the same configuration as the IDT thin film.

In one specific embodiment, the thickness of the ground thin film may vary depending on the material of the ground thin film, and may be 10 to 200 nm. If this range is exceeded, transparency cannot be secured. For example, when ITO is used as the material of the thin film, the thickness may be 100 to 200 nm, and when gold is used, the thickness may be 10 to 20 nm.

The acoustic wave generating resonator according to the present invention can generate micro-vibration and resonance based on the electrical-to-mechanical conversion phenomenon due to polarization within the non-piezoelectric dielectric polymer substrate upon application of an electric field, rather than the conventional electrical-to-mechanical conversion phenomenon due to the (inverse) piezoelectric effect.

This resonance phenomenon is a principle in which an acoustic wave is amplified as constructive interference occurs due to the matching of the wavelength corresponding to the interdigitated IDT electrode and the frequency of the applied alternating current.

In one specific embodiment, resonance may be generated in the resonator by the occurrence of constructive interference along with wave generation when an alternating current having a frequency of 1 to 100 MHz is applied.

In one specific embodiment, the resonant frequency used may vary depending on the design elements of the resonator.

In one specific embodiment, the acoustic wave generated from the resonator may promote cell migration and proliferation.

In one specific embodiment, when an alternating current having a frequency in a range of 1 to 100 MHz is applied to the resonator, an acoustic wave amplified by constructive interference may be generated.

In one specific embodiment, the resonator may have flexibility, enabling attachment to a curved surface of a human body, and have transparency, enabling real-time optical imaging of cells cultured on the device.

The present invention also relates to a method for manufacturing the aforementioned acoustic wave generating resonator.

The method for manufacturing an acoustic wave generating resonator according to the present invention comprises (S1) a step of preparing a non-piezoelectric dielectric polymer substrate;
a step of depositing an IDT thin film, comprising an Interdigital transducer (IDT) electrode and a pad electrode electrically connected to the IDT electrode, on one surface of the substrate so as to form an electric field with the substrate interposed therebetween;
(S2) a step of coating an insulating film on the one surface of the substrate so as to house the IDT thin film; and
(S3) a step of forming a ground thin film on the other surface of the substrate.

In the present invention, the resonator can be manufactured by performing (S1), (S2), and (S3) sequentially, or in the order of (S1), (S3), and (S2).

In the present invention, step (S1) is a step of depositing an IDT thin film, comprising an Interdigital transducer (IDT) electrode and a pad electrode electrically connected to the IDT electrode, on one surface of a non-piezoelectric dielectric polymer substrate.

In the present invention, before performing the deposition, a step of dicing the non-piezoelectric dielectric polymer substrate to an appropriate size (for example, 24 mm*24 mm) and then cleaning it may be additionally performed. The cleaning may be performed using Isopropyl alcohol (IPA) and/or Acetone. In addition, after cleaning, the residual solvent can be removed by vacuum evaporation.

In one specific embodiment, the IDT thin film may be formed using Physical Vapor Deposition (PVD), sputtering, metal transfer, electron beam deposition, chemical vapor deposition, atomic layer deposition, molecular beam epitaxy, or laser metal deposition, and specifically, may be deposited using Physical Vapor Deposition (PVD).

In one specific embodiment, during the deposition of the IDT thin film, a shadow mask with a shape corresponding to the IDT electrode and the pad electrode may be used to allow selective inflow of metal vapor into the IDT electrode and pad electrode patterns within the mask. In the present invention, step (S2) is a step of forming an insulating film on one surface of the substrate so as to house the IDT thin film. In this step, the insulating film can be formed through spin coating.

In the present invention, step (S3) is a step of forming a ground thin film on the other surface of the non-piezoelectric dielectric polymer substrate.

In one specific embodiment, the ground metal thin film may be formed using Physical Vapor Deposition (PVD), sputtering, metal transfer, electron beam deposition, chemical vapor deposition, atomic layer deposition, molecular beam epitaxy, or laser metal deposition, similarly to step (S1), and specifically, may be deposited using Physical Vapor Deposition (PVD).

The acoustic wave generating resonator for cell stimulation for acoustic wave generation according to the present invention can be manufactured as a wearable patch and can be used by being attached to the skin or tissue.

In addition, the present invention relates to an in vitro method for cell stimulation using the aforementioned acoustic wave generating resonator.

In an embodiment of the present invention, the resonance phenomenon at a specific frequency was confirmed through characterization of the wave generated from the resonator, and by applying this to cells, an improved cell migration speed and proliferation rate were confirmed. The wave generated from the resonator according to the present invention can mechanically stimulate cells, and this can promote cell division and migration.

Hereinafter, the advantages and features of the present invention, and the methods for achieving them will become clear with reference to the examples. However, the present invention is not limited to the examples disclosed below, but can be implemented in various forms, and the scope of the present invention is not limited by the examples presented below.

### EXAMPLES

### Preparation Example 1. Manufacture of an Acoustic Wave Generating Resonator using Polyimide as a Non-Piezoelectric Dielectric Polymer

(1) Non-Piezoelectric Dielectric Polymer Substrate Fabrication Polyimide Kapton®(DuPont) was diced to a size of 24 mm×24 mm, then sequentially cleaned with isopropyl alcohol (Duksan Chemical) and acetone (Duksan Chemical), and then subjected to vacuum evaporation for 12 hours.

### (2) IDT Thin Film Deposition

A metal thin film was deposited on one surface of the non-piezoelectric dielectric polymer substrate prepared in (1) through physical vapor deposition (thermal evaporation).

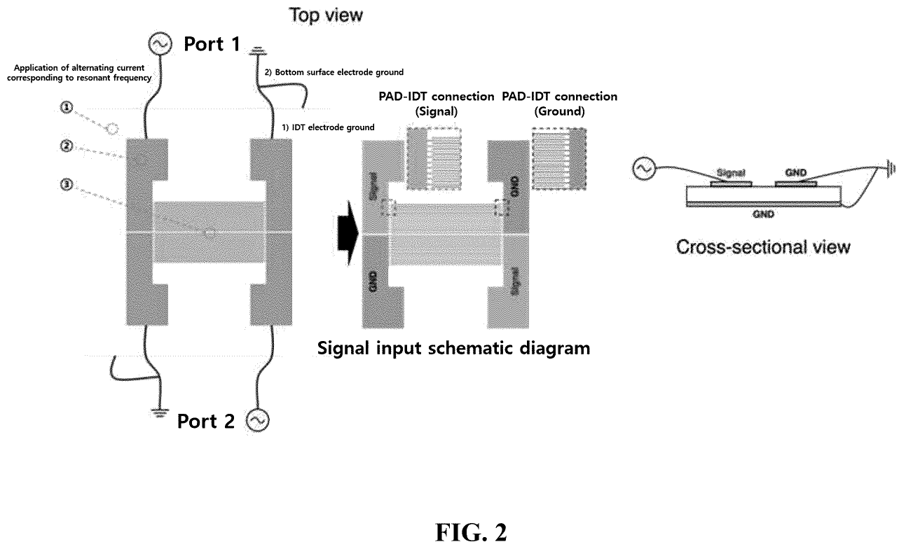

A shadow mask (Yesung) corresponding to the IDT electrode and pad electrode was placed on one surface of the substrate, and then inserted into a Thermal Evaporation System (DaeDong Hightechnologies) chamber. At this time, the design of the deposited electrode corresponds to FIG. 2.

Electrode deposition was carried out in a high vacuum state of 6.0×10⁻⁶ Torr as follows; chromium and gold were deposited to 3 and 17 nm at speeds of 0.1 Å/sec and 0.3 Å/sec, respectively, and at this time, the holder was rotated at an appropriate RPM for uniform pattern formation. Although the pressure inside the evaporator may increase slightly during the thermal evaporation process, this does not affect the uniformity of the electrode pattern.

### (3) Ground Thin Film Deposition

A metal thin film was deposited on the other surface of the non-piezoelectric dielectric polymer substrate.

Physical vapor deposition was performed under the same deposition conditions as in (2), except that no shadow mask was used.

### (4) PDMS Insulating Film Layer Spin Coating

The base and curing agent of a Sylgard 184 silicone elastomer kit (SylgardTM) were mixed at a ratio of 10:1, and then bubbles were removed for 2 hours. This was coated on the non-piezoelectric dielectric polymer substrate on which the IDT thin film prepared in (3) was formed, and then spin-coated at 1150 RPM for 60 seconds.

The PDMS-coated resonator was cured in an 80° C. oven for 2 hours to complete the insulating film coating.

### Experimental Example 1: Characteristic 1 of the Acoustic Wave Generating Resonator Fabricated in Preparation Example 1

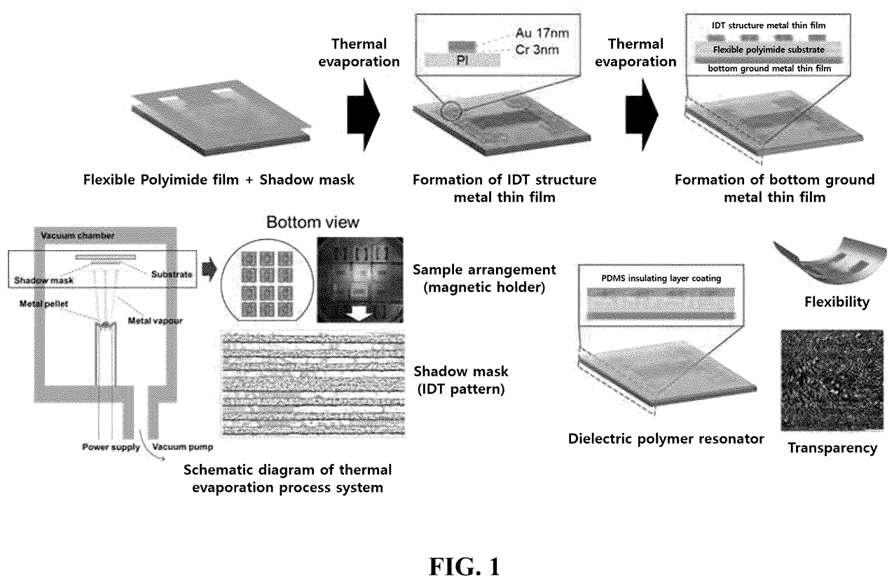

FIG. 1 shows the process of manufacturing the acoustic wave generating resonator. In addition, FIG. 2 shows a schematic diagram of the manufactured acoustic wave generating resonator. In FIG. 2, \textcircled{1} represents a substrate, \textcircled{2} represents a pad electrode, and \textcircled{3} represents an IDT electrode.

The acoustic wave generating resonator according to the present invention can be manufactured in the order of Preparation Example 1. The IDT electrodes with an interdigitated structure play a pivotal role in wave generation and have a form in which the fingers of a signal (alternating current) electrode and a ground electrode (GND) are interdigitated (FIG. 2). The acoustic wave generating resonator according to the present invention is based on the principle that resonance occurs as constructive interference occurs along with wave generation when an alternating current with a specific frequency is applied to the repeating pattern of signal and ground electrodes.

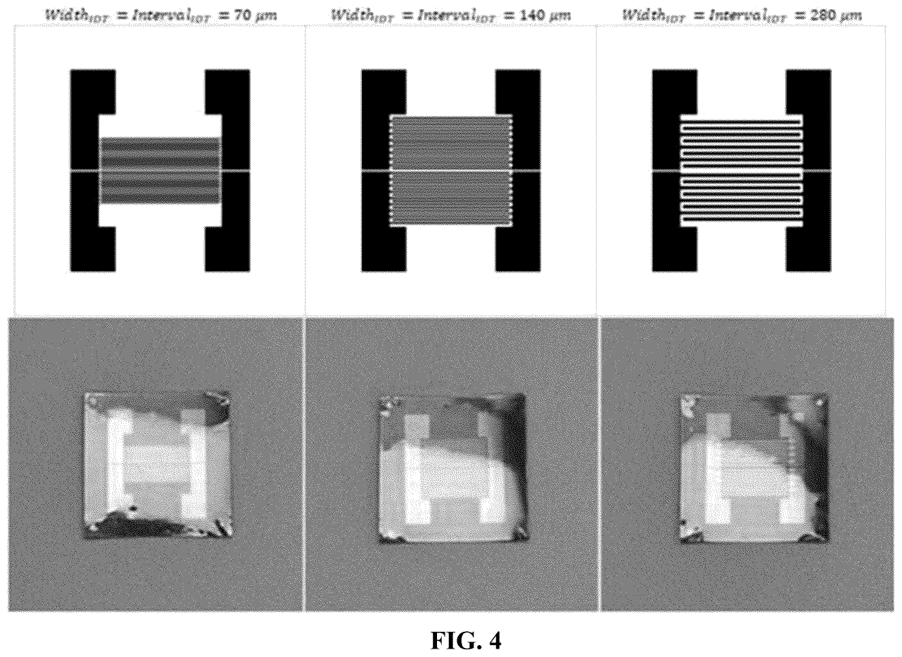

In the present invention, FIGS. 3 and 4 show a schematic diagram of the IDT thin film and the IDT thin film manufactured according to an embodiment of the present invention. In the present invention, IDT thin films of various structures can be manufactured, and acoustic wave generation can be optimized.

### Experimental Example 2: Characteristic 2 of the Acoustic Wave Generating Resonator Fabricated in Preparation Example 1

For stable acoustic wave generation, a uniformly deposited IDT electrode is important. To achieve this, a high level of resolution within the electrode pattern formed during the IDT electrode deposition must be secured from Batch-to-Batch.

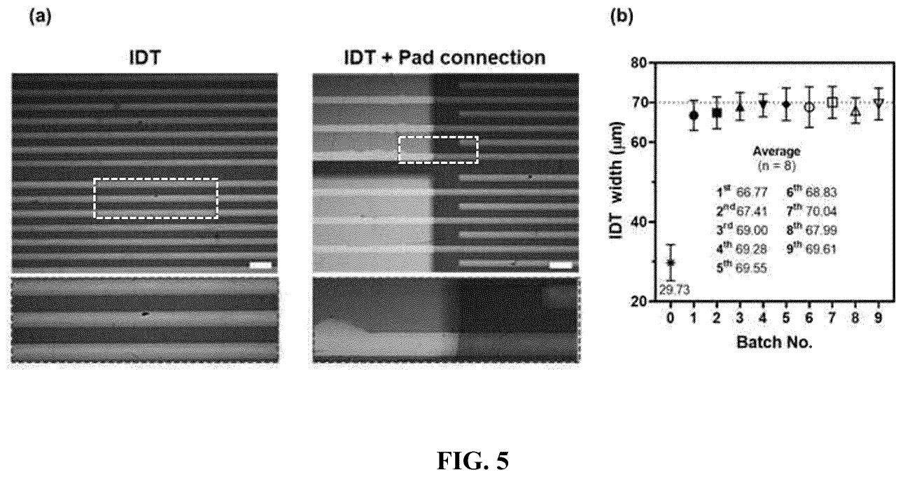

The appearance of the IDT thin film deposited through the optimized process as in (2) of Preparation Example 1 was photographed using an optical microscope, and the result is shown in FIG. 5(a). And, the width of one finger of the deposited IDT electrode was measured through ImageJ software (National Institute of Health, USA), and shown in FIG. 5(b).

As shown in FIG. 5, the width of the IDT electrode is about 70 μm, and it can be confirmed that the electrode widths deposited in Batches 1 to 9 all reach the target value. At this time, Batch 0 represents the measurement data before process optimization, and when comparing this with the data after optimization, it can be confirmed that the electrode deposition process used in the present invention shows excellent resolution and reproducibility.

Experimental Example 2: Characteristic 2 of the Acoustic Wave Generating Resonator Fabricated in Preparation Example 1

The final configuration of the acoustic wave generating resonator includes the following: ground thin film-non-piezoelectric dielectric polymer substrate-IDT thin film-insulating film (FIG. 1).

In this experimental example, the non-piezoelectric dielectric polymer substrate of the resonator used in the cell experiment is a 25 μm thick polyimide, which has a level of transparency that allows for real-time imaging.

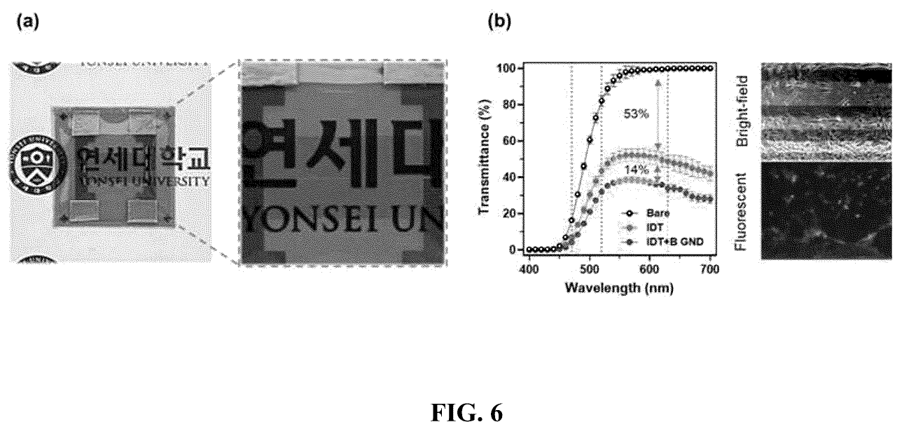

The transparency of the acoustic wave generating resonator manufactured in Preparation Example 1 was confirmed qualitatively and quantitatively, and the results are shown in FIG. 6(a) and FIG. 6(b), respectively. The quantitative analysis of the transmittance of the resonator in the visible light spectrum was performed by measuring the absorbance in the spectrum using a microplate reader (SpectraMax ABS Plus; Molecular Devices, San Jose, CA, USA) and then converted based on the following Beer-Lambert law equation:

Transmittance (%)=10^{2-Absorbance}

Specifically, FIG. 6(a) is an image transmitted through the device, and it can be confirmed that the resonator of this experimental example is transparent.

In addition, the graph of FIG. 6(b) is a spectrum analysis graph of visible light region transmittance, where Bare represents the transmittance of a substrate without a metal thin film (electrode), IDT represents the transmittance of a substrate with an IDT thin film formed on one surface, and IDT+B GND represents the transmittance of a substrate with an IDT thin film formed on one surface and a ground thin film formed on the other surface. In addition, the photos above and below show optical microscope images of cells seeded on the device. Due to the yellow characteristic of polyimide, it showed low transmittance in the purple-blue region of the visible light spectrum, but it was confirmed that there was no problem with imaging. In addition, it was confirmed that it showed excellent transmittance even during real-time cell imaging through an optical/fluorescence microscope.

### Experimental Example 4: Characteristic 4 of the Acoustic Wave Generating Resonator Fabricated in Preparation Example 1

The acoustic wave generating resonator manufactured in Preparation Example 1 is fabricated based on a flexible substrate. Due to this characteristic, it can be smoothly attached to a curved body surface. At this time, deformation (bending) in the device caused by the curvature of the attachment surface may cause damage to the electrode, but by coating it with a PDMS insulating film, the tensile and compressive forces applied to the IDT electrode during deformation can be minimized.

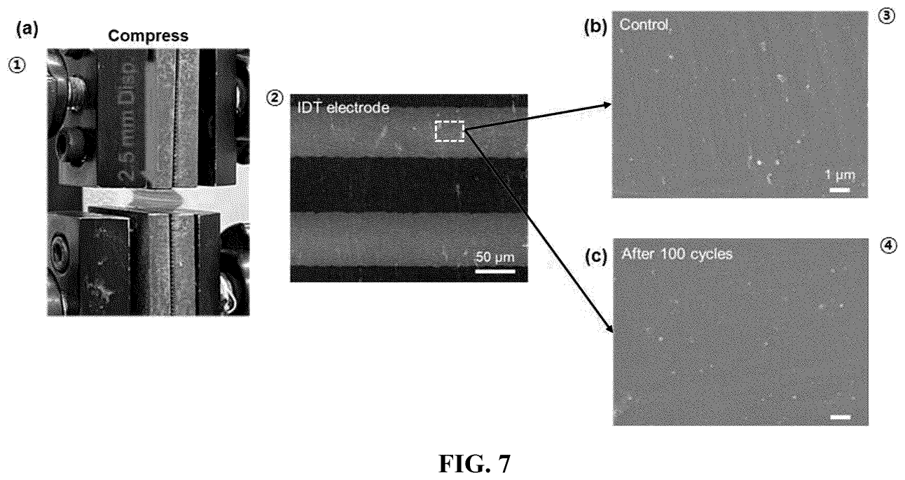

In this experimental example, to verify this, a device deformation test was conducted using a Universal Testing Machine (UTM) (Mecmesin, Slinfold, West Sussex, United Kingdom), and then the surface was observed through an electron microscope (JEOL-IT500HR, JEOL Co., Tokyo, Japan). The results are shown in FIG. 7.

Specifically, in FIG. 7, \textcircled{1} is a photograph of the resonator to which a deformation of 2.5 mm is applied by a UTM, in 7, \textcircled{2} is an electron micrograph of the IDT electrode (low magnification), \textcircled{3} is an electron micrograph of the IDT electrode (high magnification/before deformation experiment-control group), and \textcircled{3} is an electron micrograph of the IDT electrode (high magnification/after deformation experiment-experimental group).

As shown in FIG. 7, when the electrode surfaces were photographed before/after performing 100 cycles of applying a 2.5 mm deformation to the acoustic wave generating resonator, it can be confirmed that there is no electrode damage due to the deformation of the device.

### Experimental Example 5: Acoustic wave Characteristic 1 of the Acoustic Wave Generating Resonator Fabricated in Preparation Example 1

The acoustic wave generated from the IDT electrode undergoes constructive interference at a specific frequency, and due to this, it can have an amplified intensity. At this time, among the frequencies of the applied alternating current, the range that can cause the above constructive interference was set as the resonant frequency. To verify this, a fluorescently treated polystyrene particle tracking experiment was conducted as follows.

Nile red-colored FluoSpheresTM carboxylate-microspheres (Thermo Fisher Scientific) with a diameter of 2 μm were diluted 100-fold in distilled water, and a droplet of 3 μm was dropped on the center of the IDT electrode. At this time, the polystyrene beads in the droplet can rotate within the droplet due to the acoustic wave generated from the resonator.

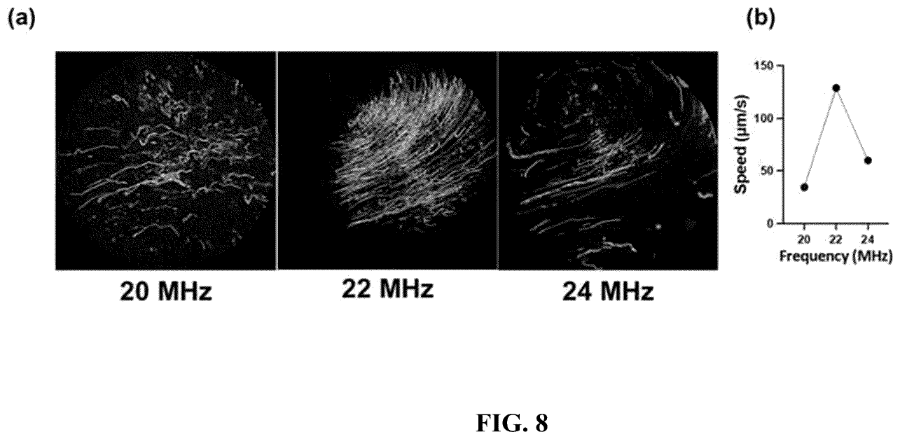

FIG. 8 is data confirming the resonance phenomenon of the acoustic wave generated from the resonator. FIG. 8(a) shows the tracking path of the rotating polystyrene beads according to the applied frequency, and it shows different movement paths according to the frequency of the applied alternating current. Through the figure, it can be confirmed that at 22 MHz, it rotated strongly with a uniform path compared to the surrounding frequencies. In addition, FIG. 8(b) is a plot of the bead's streaming velocity analysis, and it can be confirmed that the rotation speed at 22 MHz is significantly faster than at 20 and 24 MHz. This indicates that the acoustic wave generating resonator used in the present invention resonates at a frequency of 22 MHz.

### Experimental Example 6: Acoustic Wave Characteristic 2 of the Acoustic Wave Generating Resonator Fabricated in Preparation Example 1

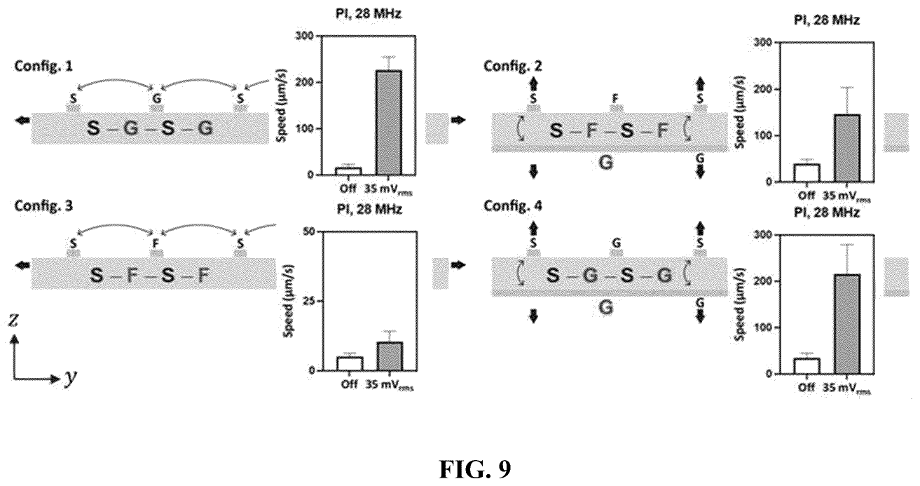

A bead tracking experiment according to the electrode connection structure was conducted in the same manner as described in Experimental Example 5. The results are shown in FIG. 9.

What each electrode structure represents is as follows; Structure 1) IDT ground/bottom surface no ground, 2) IDT no ground/bottom surface ground, 3) IDT no ground/bottom surface no ground, 4) IDT ground/bottom surface ground. When the velocity of the beads rotating due to the acoustic wave generated in each structure was quantitatively analyzed, it can be confirmed that the presence or absence of a ground within the IDT electrode determines the generation of the acoustic wave.

### Preparation Example 2. Manufacture of an Acoustic Wave Generating Resonator Using Polyethylene Terephthalate (pet) as a Non-piezoelectric Dielectric Polymer

An acoustic wave generating resonator was manufactured in the same manner as in Preparation Example 1, except that a polyethylene terephthalate (Kolon Industries) sheet was used instead of polyimide as the non-piezoelectric dielectric polymer.

### Preparation Example 3: Manufacture of an Acoustic Wave Generating Resonator using PDMS as a Non-Piezoelectric Dielectric Polymer (Applying Metal Transfer)

Metal transfer was carried out according to the process in Microsyst Technol (2014) 20:1783-1789.

A silicon wafer (Nam-gang High-tech Co., Ltd.) was diced to a size of 24 mm×24 mm, and the surface was cleaned with isopropyl alcohol and acetone.

Subsequently, after depositing an electrode in the same manner as the electrode deposition method described in Preparation Example 1, it was treated in a 25 mM solution of 3-Mercaptopropyl trimethoxysilane (MPTMS, Sigma-Aldrich) for 30 minutes to attach a methoxy functional group to the electrode. This was performed to increase the bonding force by introducing a covalent bond between the PDMS elastomer substrate and the electrode.

The wafer-electrode-MPTMS composite was washed with 95% ethanol (Duksan Chemical), and then a PDMS precursor solution was spin-coated at 1150 RPM for 60 seconds to coat about 50 micrometers thick PDMS, followed by curing in an 80° C. oven for 2 hours. This was immersed in an isopropyl alcohol solution, and the PDMS was peeled off from the wafer to complete the fabrication of the PDMS resonator with the deposited electrode.

### Comparative Preparation Example 1. Manufacture of an Acoustic Wave Generating Resonator using PVDF as a Piezoelectric Element

An acoustic wave generating resonator was manufactured in the same manner as in Preparation Example 1, except that PVDF, a piezoelectric element, was used instead of polyimide as the non-piezoelectric dielectric polymer.

### Experimental Example 7: Acoustic Wave Characterization

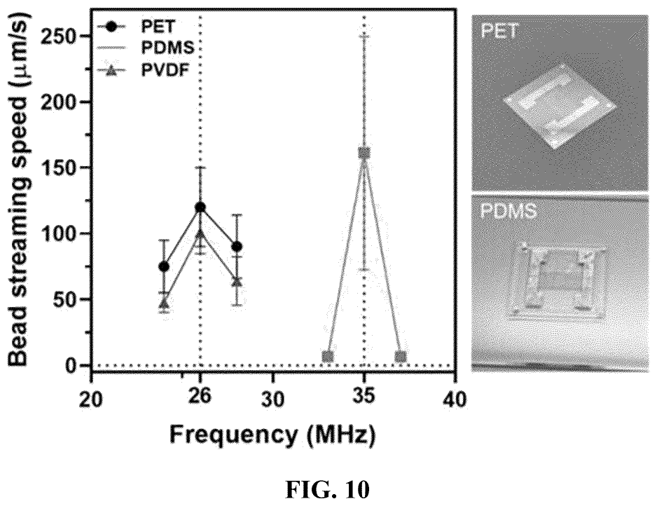

The acoustic wave generation aspect when a non-piezoelectric dielectric polymer other than polyimide was used in the manufacture of the resonator of the present invention was experimented in the same manner as in Example 5, and the results are shown in FIG. 10. At this time, resonators were manufactured using PET (Preparation Example 2), PDMS (Preparation Example 3), and the piezoelectric polymer PVDF (Comparative Preparation Example 1) as substrate materials, and the PVDF corresponds to a piezoelectric element as a control group.

As shown in FIG. 10, the PET, PDMS, and PVDF-based resonators showed resonant frequencies of 26, 35, and 26 MHz, respectively, and it can be confirmed that the non-piezoelectric polymers PET and PDMS generated acoustic waves of stronger intensity compared to the piezoelectric polymer PVDF.

### Experimental Example 8: Cell Toxicity Experiment

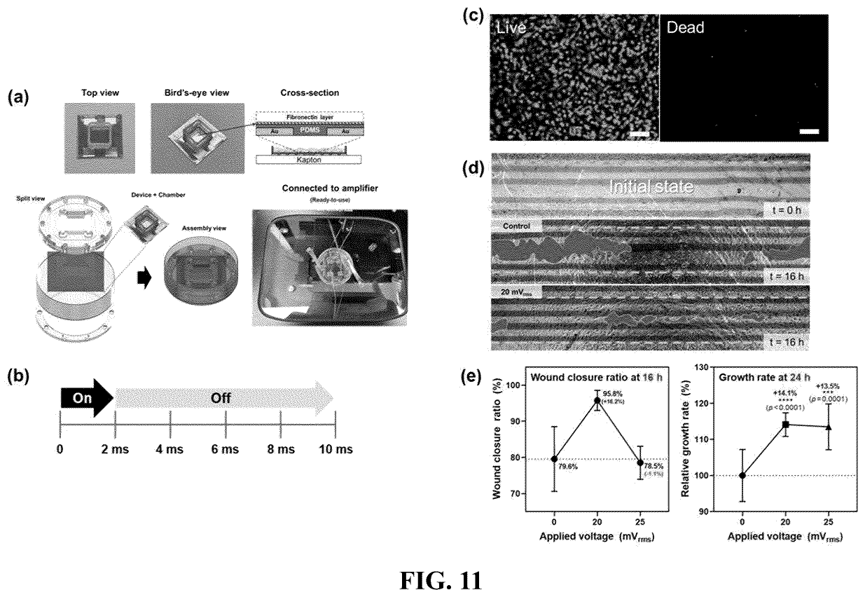

For the cell experiment of the acoustic wave generating resonator manufactured in Preparation Example 1, a cell experiment platform was fabricated as shown in FIG. 11(a).

Specifically, in FIG. 11(a), the top photo shows a photograph of the resonator with a PDMS chamber attached for the cell experiment, and the right diagram shows a schematic diagram of Fibronectin protein coating for smooth cell attachment. In addition, the bottom photo shows a device holder schematic diagram for cell experiment and imaging (TPU) (left) and the cell experiment set-up with wire connection and real-time imaging device combination (right).

The cells used were NIH-3T3 fibroblasts (ATCC, Rockville, MD, USA), which were seeded on the surface of a resonator coated with 25 μg/mL of fibronectin protein (Gibco, Waltham, MA, USA). The cells were stimulated by a pulsed wave for 5 minutes a day with a 20% duty ratio, and at this time, as described in FIG. 11(b), the resonator was operated for only 2 ms out of a 10 ms cycle, and remained in a standby state for 8 ms.

To confirm the cell toxicity when stimulating cells with this method, Calcein-AM (Thermo Fisher Scientific, Waltham, MA, USA) and Ethidium homodimer-1 (EthD-1, Thermo Fisher Scientific, Waltham, MA, USA) fluorescence staining (hereinafter Live/Dead assay) was performed.

As shown in FIG. 11(c), it can be confirmed that the acoustic wave generated from the acoustic wave generating resonator according to the present invention is not toxic to cells.

### Experimental Example 9: Cell Migration/Proliferation Experiment

To evaluate the wound healing ability of the acoustic wave generated from the acoustic wave generating resonator manufactured in Preparation Example 1, an artificial wound with a width of 500 μm was formed, and then cells were stimulated in the same manner as described in Experimental Example 8.

Afterward, the wound healing rate for 16 hours and the cell proliferation rate for 24 hours were analyzed as shown in 11(d) and FIG. 11(e).

In FIG. 11(d), the three photos from the top show a photo of an artificial wound (width=500 μm) formed using NIH-3T3 cells, a photo of the control group without applying acoustic waves (the colored area means a cell-free area), and a photo of the group that generated acoustic waves with an intensity of 20 mV. In addition, in FIG. 11(e), the left photo shows the wound healing rate analysis result (t=16 h), and the right photo shows the cell proliferation rate analysis result (t=24 h).

As shown in FIG. 11(d) and (e), it can be confirmed that when an acoustic wave was generated at 20 mV, a 16.2% improved wound healing rate and a 14.1% improved cell proliferation rate were shown compared to the control group.

### Experimental Example 10: Analysis of Resonant Frequency Change According to the Width of the Finger

When manufacturing the resonator of the present invention, the width of the finger was varied, and the acoustic wave generation aspect according to the finger width was experimented in the same manner as in Example 5.

Specifically, an acoustic wave generating resonator using polyimide was manufactured according to the method of Preparation Example 1, and the average width of the finger was manufactured to be 70 μm, 140 μm, and 210 μm. The structure of the manufactured IDT thin film is as shown in FIG. 4.

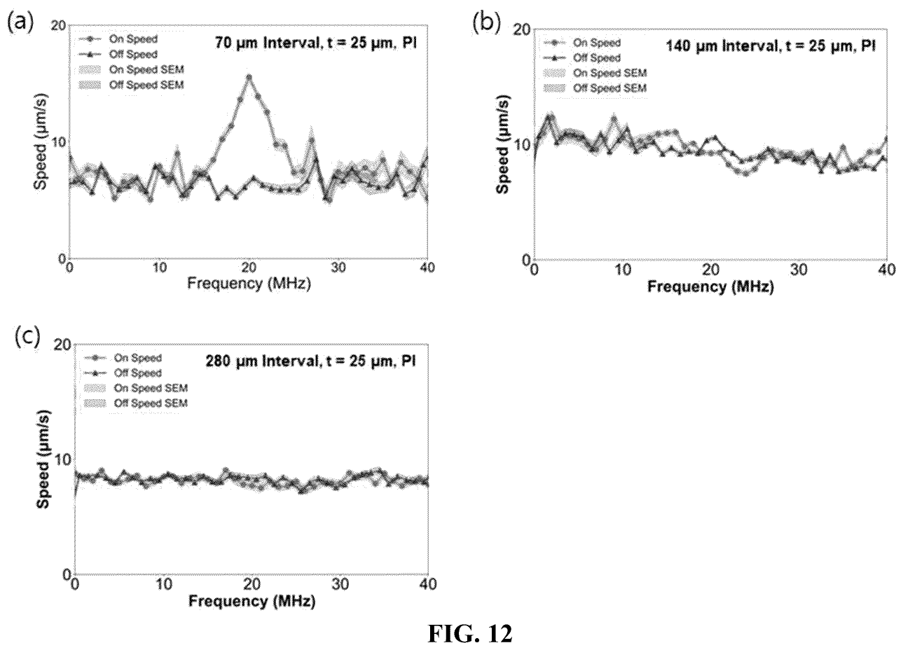

The results of the resonant frequency change analysis are shown in FIG. 12.

The resonant frequency experiment was conducted by shifting the frequency of the applied alternating current by 1 MHz in the range of 1-40 MHz. As shown in FIG. 12, it can be confirmed that the most noticeable resonance aspect is shown when the finger width is 70 μm (the most intensive wave is generated at 20 MHz), and at other finger widths, a phenomenon presumed to be resonance is not observed. Through this, it can be confirmed that the preferred average width of the finger is 70 μm.

What is claimed is:

1. A resonator for cell stimulation for acoustic wave generation, comprising:
a non-piezoelectric dielectric polymer substrate;
an IDT thin film formed on one surface of the substrate and comprising an Interdigitated transducer (IDT) electrode and a pad electrode electrically connected to the IDT electrode;
an insulating film formed on the one surface of the substrate and housing the IDT thin film therein; and
a ground thin film formed on the other surface of the substrate,
wherein an electric field is formed across the substrate by an alternating current voltage applied between the IDT thin film and the ground thin film, and polarization of the substrate induced by the electric field causes mechanical deformation to generate an acoustic wave.

2. The resonator of claim 1, wherein a material of the non-piezoelectric dielectric polymer substrate is Polyimide(PI), Polyethylene terephthalate(PET), Polydimethylsiloxane (PDMS), or Polyurethane (PU).

3. The resonator of claim 1, wherein a thickness of the non-piezoelectric dielectric polymer substrate is 5 to 150 μm.

4. The resonator of claim 1, wherein the IDT thin film is composed of a metal or a conductive polymer,
the metal is one or more selected from the group consisting of gold (Au), copper (Cu), aluminum (Al), and Indium Tin Oxide (ITO), and
the conductive polymer is PEDOT: PSS.

5. The resonator of claim 1, wherein a thickness of the IDT thin film is 10 to 200 nm.

6. The resonator of claim 1, wherein the IDT electrode and the pad electrode are respectively composed of a signal electrode and a ground electrode,
the signal electrode of the IDT electrode is electrically connected to the signal electrode of the pad electrode, and
the ground electrode of the IDT electrode is electrically connected to the ground electrode of the pad electrode.

7. The resonator of claim 6, wherein the IDT electrode and the pad electrode respectively exist as an even number of two or more, and
the IDT electrode and the pad electrode exist in the same number.

8. The resonator of claim 6, wherein, in the IDT electrode, one signal electrode comprises one or more fingers,
one ground electrode comprises one or more fingers, and
one finger of the signal electrode has a structure interdigitated with one finger of the ground electrode.

9. The resonator of claim 8, wherein a length of the one finger is 5 to 15 mm, and a width is 50 to 100 μm.

10. The resonator of claim 8, wherein a distance between the one finger and an adjacent finger is 30 to 500 μm.

11. The resonator of claim 1, wherein the insulating film comprises one or more selected from the group consisting of Polydimethylsiloxane (PDMS), Polyurethane (PU), and Silicone elastomer.

12. The resonator of claim 1, wherein the ground thin film is composed of a metal or a conductive polymer,
the metal is one or more selected from the group consisting of gold (Au), copper (Cu), aluminum (Al), and ITO, and
the conductive polymer is PEDOT: PSS.

13. The resonator of claim 1, wherein a thickness of the ground thin film is 10 to 200 nm.

14. The resonator of claim 1, wherein resonance is generated by the occurrence of constructive interference along with wave generation when an alternating current having a frequency of 1 to 100 MHz is applied to the resonator.

15. The resonator of claim 1, wherein the acoustic wave generated from the resonator promotes cell migration and proliferation.

16. The resonator of claim 1, wherein when an alternating current having a frequency in a range of 1 to 100 MHz is applied, an acoustic wave amplified by constructive interference is generated.

17. The resonator of claim 1, wherein the resonator has flexibility, enabling attachment to a curved surface of a human body, and has transparency, enabling real-time optical imaging of cells cultured on the device.

18. A method for manufacturing an acoustic wave generating resonator, comprising:
a step of preparing a non-piezoelectric dielectric polymer substrate;
a step of depositing an IDT thin film, comprising an Interdigital transducer (IDT) electrode and a pad electrode electrically connected to the IDT electrode, on one surface of the substrate so as to form an electric field with the substrate interposed therebetween;
a step of coating an insulating film on the one surface of the substrate so as to house the IDT thin film therein; and
a step of depositing a ground thin film on the other surface of the substrate.

19. An in vitro method for cell stimulation using the acoustic wave generating resonator for acoustic wave generation according to claim 1.
